(19) United States
(12) Patent Application Publication
(10) Pub. No.: US 2022/0096363 A1
(43) Pub. Date: Mar. 31, 2022

(54) Oral Care Composition Comprising Hops And Flavor

(71) Applicant: The Procter & Gamble Company, Cincinnati, OH (US)

(72) Inventors: Arif Ali Baig, Mason, OH (US); Tammy K. Baker, Cincinnati, OH (US); Pierig Jean-Marie Lepont, Wyoming, OH (US); Samuel James St. John, Cincinnati, OH (US)

(21) Appl. No.: 17/485,553

(22) Filed: Sep. 27, 2021

Related U.S. Application Data
(60) Provisional application No. 63/084,018, filed on Sep. 28, 2020.

(51) Int. Cl. A61K 8/9789; A61K 8/37; A61Q 11/00

(52) U.S. Cl. CPC A61K 8/9789; A61K 8/37; A61K 2800/28; A61K 2800/5922; A61Q 11/00

(57) ABSTRACT

Oral care compositions including hops and up to about 0.20% of aromatic ester flavor. Oral care compositions including hops and up to about 0.20% of methyl salicylate. Fluoride-free toothpaste compositions including hops and up to about 0.20% of methyl salicylate. Fluoride-free toothpaste compositions including hops and less than about 0.20% of methyl salicylate.

### FIELD OF THE INVENTION

The present invention is directed to compositions comprising hops, such as hops extracts, hops alpha acid, and/or hops beta acid, and flavor. The present invention is also directed to compositions comprising two compounds with incompatible taste profiles, such as hops and flavor compounds including an aromatic ester.

### BACKGROUND OF THE INVENTION

Natural compounds with antibacterial activity, such as hops, can be incorporated into oral care compositions to provide antibacterial and/or anticavity activity. Natural antibacterial agents, such as hops, can include mixtures of active compounds, oils, flavonoids, and/or other flavor compounds. However, many natural antibacterial agents can have a strong, unpleasant, and/or bitter taste when used at the amounts necessary to receive the antibacterial benefit. Thus, many natural antibacterial agents can be unsuitable for use in oral care compositions despite having high antibacterial activity.

One solution to the unpalatable taste of many natural antibacterial agents could be the use of flavors to mask or hide the taste of natural antibacterial agents, such as hops. Unfortunately, the combination of two or more distinct tastes can also be unpalatable. Thus, there is a need for oral care compositions that include natural antibacterial agents but remain palatable for use in oral care compositions.

### SUMMARY OF THE INVENTION

Disclosed herein is an oral care composition comprising: (a) hops; and (b) flavor, wherein the flavor comprises up to about 0.20%, by weight of the oral care composition, of aromatic ester. Also disclosed herein is an oral care composition comprising: (a) hops; and (b) flavor, wherein the flavor comprises up to about 0.20%, by weight of the oral care composition, of methyl salicylate.

Also disclosed herein is an oral care composition comprising: (a) hops, such as hops extract and/or hops beta acid; and (b) flavor, wherein the flavor comprises up to about 0.20%, by weight of the oral care composition, of aromatic ester.

Also disclosed herein is an oral care composition comprising: (a) hops; and (b) flavor, wherein the flavor comprises up to about 0.20%, by weight of the oral care composition, of wintergreen.

### DETAILED DESCRIPTION OF THE INVENTION

Oral care compositions are formulated with distinctive flavors. Typically, oral care compositions comprise a collection of distinct flavor compounds, which each contribute to the overall taste of the composition. Flavors encourage the use of the oral care composition so that the user can receive benefits from oral care actives within the oral care composition. However, unless the taste of the composition is palatable, the user will not use the composition nor derive the benefit from the composition's use. Therefore, the effort to pleasingly flavor an oral care composition is neither trivial nor accidental. Many natural, plant-derived antibacterial agents have such distinct flavors that the conventional approach to oral care flavoring is not sufficient. In some cases, the combination of natural and/or plant-derived antibacterial agents with common flavor compounds can result in an unpalatable composition.

Why do two things that taste good on their own sometimes taste so bad when taken together? As omnivores, humans have historically faced the difficult task of identifying and gathering food that satisfies nutritional needs while avoiding foodborne illnesses and have refined their taste/olfactory palate to support this task. Although many factors such as color, texture, temperature, and sound play an important role in oral sensation, palatability is largely determined by flavor, representing a group of sensations including odors (due to molecules that can bind olfactory receptors), tastes (due to molecules that stimulate taste buds), and freshness or pungency (trigeminal senses). The complicated interplay between these chemical profiles (flavors) makes identification of complementary and incompatible flavors unexpectedly challenging.

Among the five tastes, salty, sweet, and umami are appetitive (driving us toward essential nutrients), whereas bitter and sour are aversive (alerting us to potentially harmful substances). Mixing aversive tastes with appetitive tastes sends conflicting information to the brain, and this type of confusion is what the senses are trying to avoid. The mixed signal is why people reject food that has gone bad. There can be delight in confusion, such as sweet and sour Chinese food, but generally conflicting tastes can lead to negative reactions, such as potentially cocoa and pickles, peanut butter on a hot dog, or even soy sauce in milk.

One strategy to overcome unpalatable flavor can be to overwhelm the senses, such as the addition of sweeteners. Active agents used as medicines can be poisonous at high doses, and can have a bitter taste on their own. As such, the bitter taste of many active agents can be made more palatable by camouflaging with sugar. However, the addition of sugar can be unpreferable in dental compositions due its contribution to the development of cavities.

Additionally, it is not just a question of the flavor compound itself but also the concentration of the flavor compound. For example, consider the idea of dairy turning rancid. Dairy rancidity is caused by the oxidation of fatty acids to butyric acid, which produces a unique smell. At low levels, butyric acid can be pleasant and highly prized (e.g., Parmesan cheese). However, at high levels, butyric acid can be extremely unpalatable, such as in human vomit. Thus, there is a very thin line between palatable and unpalatable.

Unfortunately, researchers have tried and failed to determine simple rules for the combination of flavors that are palatable/unpalatable. Instead, the process is best suited to discovery.

The three primary mint flavor families (i.e., peppermint, spearmint, and wintergreen) typically used in oral care compositions have largely chemically distinct flavor profiles. Peppermint oil is primarily composed of menthol, menthone, and menthyl acetate and contains low amounts of the principle chemical ingredients of spearmint oil and wintergreen oil. The principle chemical ingredient giving spearmint oil its distinctive flavor are carvone and limonene with low levels of the principle components of peppermint or wintergreen. Whereas, wintergreen oil is primarily composed of methyl salicylate.

While not wishing to be bound by theory, it is believed that the combination of hops bitter acids and methyl salicylate results in a uniquely unpalatable experience due at least in part to the bitterness of the hops being amplified instead of masked. Additionally, this combination of hops and methyl salicylate leads to a sour and unpleasant experience for some users. Thus, only a minimal amount of methyl salicylate can be used in combination with hops in oral care compositions.

It was further determined that peppermint- and spearmint-based flavor systems can be combined with hops in oral care compositions to create palatable tastes. In general, this principle can guide the development of palatable oral care compositions comprising hops.

Unpalatable flavors have been observed in combination with noticeable levels of methyl salicylate, a primary component of wintergreen and chemically distinct from peppermint and spearmint. As such, the present invention is directed towards oral care compositions comprising hops with less than a noticeable amount of methyl salicylate.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, toothpaste, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such.

The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The oral care composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition. The oral care composition can include a variety of active and inactive ingredients, such as, for example, but not limited to a hops extract, a tin ion source, a calcium ion source, water, a fluoride ion source, zinc ion source, one or more polyphosphates, humectants, surfactants, other ingredients, and the like, as well as any combination thereof, as described below.

Section headers are provided below for organization and convenience only. The section headers do not suggest that a compound cannot be within more than one section. In fact, compounds can fall within more than one section. For example, stannous chloride can be both a tin ion source and a biofilm modifier, stannous fluoride can be both a tin ion source and a fluoride ion source, glycine can be an amino acid, a buffering agent, and/or a biofilm modifier, among numerous other compounds that can fit amongst several categories and/or sections.

Humulus lupulus

The oral care compositions of the present invention comprises hops. The hops can comprise at least one hops compound from Formula I and/or Formula IV. The compound from Formula I and/or Formula IV can be provided by any suitable source, such as an extract from Humulus lupulus or Hops, Humulus lupulus itself, a synthetically derived compound, and/or salts, prodrugs, or other analogs thereof. The hops extract can comprise one or more hops alpha acids, one or more hops iso-alpha acids, one or more hops beta acids, one or more hops oils, one or more flavonoids, one or more solvents, and/or water. Suitable hops alpha acids (generically shown in Formula I) can include humulone (Formula II), adhumulone, cohumulone, posthumulone, prehumulone, and/or mixtures thereof. Suitable hops iso-alpha acids can include cis-isohumulone and/or trans-isohumulone. The isomerization of humulone into cis-isohumulone and trans-isohumulone can be represented by Formula III.

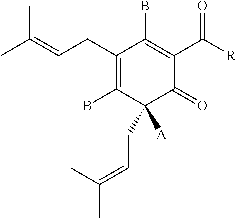

Formula I. Hops Alpha Acids. A is the acidic hydroxyl functional group in the alpha position, B are the acidic hydroxyl functional groups in the beta position, and R is an alkyl functional group.

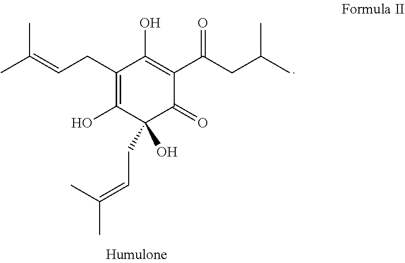 [SMILES: CC(C)=CCC1=C(O)[C@@](O)(CC=C(C)C)C(=O)C(C(=O)CC(C)C)=C1O]

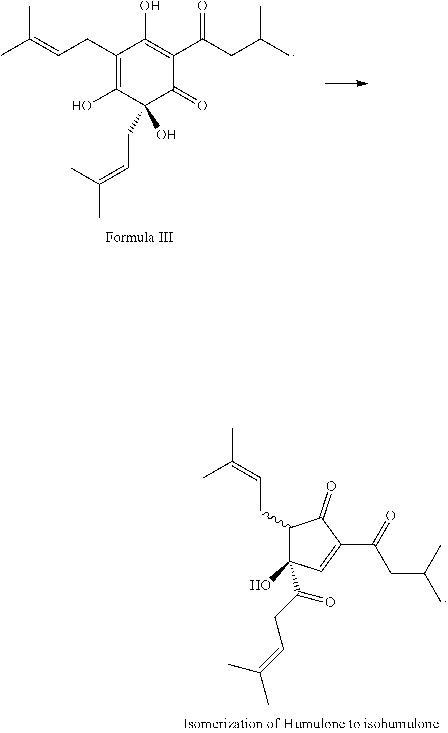

Suitable hops beta acids can include lupulone, adlupulone, colupulone, and/or mixtures thereof. A suitable hops beta acid can include a compound a described in Formula IV, V, VI, and/or VII.

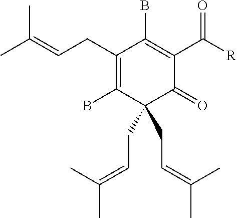

Formula IV. Hops Beta Acids. B are the acidic hydroxyl functional groups in the beta position and

R is an alkyl functional group.

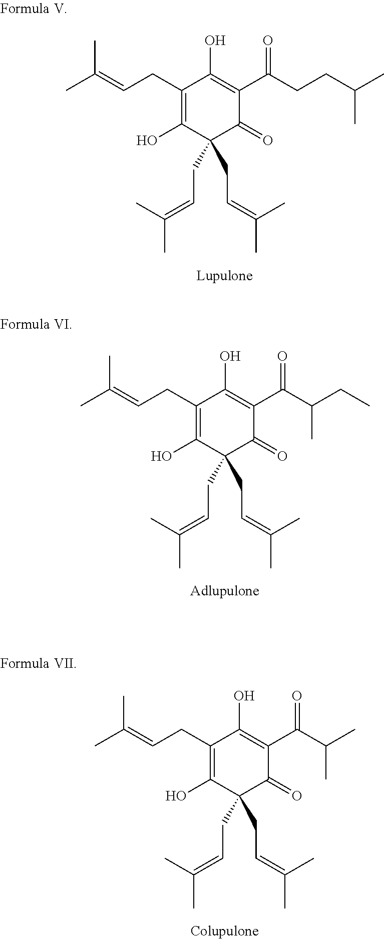

While hops alpha acids can demonstrate some antibacterial activity, hops alpha acids also have a bitter taste. The bitterness provided by hops alpha acids can be suitable for beer, but are not suitable for use in oral care compositions. In contrast, hops beta acids can be associated with a higher antibacterial and/or anticaries activity, but not as bitter a taste. Thus, a hops extract with a higher proportion of beta acids to alpha acids than normally found in nature, can be suitable for use in oral care compositions for use as an antibacterial and/or anticaries agent.

A natural hops source can comprise from about 2% to about 12%, by weight of the hops source, of hops beta acids depending on the variety of hops. Hops extracts used in other contexts, such as in the brewing of beer, can comprise from about 15% to about 35%, by weight of the extract, of hops beta acids. The hops extract desired herein can comprise at least about 35%, at least about 40%, at least about 45%, from about 35% to about 95%, from about 40% to about 90%, or from about 45% to about 99%, of hops beta acids. The hops beta acids can be in an acidic form (i.e. with attached hydrogen atom(s) to the hydroxl functional group(s)) or as a salt form.

A suitable hops extract is described in detail in U.S. Pat. No. 7,910,140, which is herein incorporated by reference in its entirety. The hops beta acids desired can be non-hydrogenated, partially hydrogenated by a non-naturally occurring chemical reaction, or hydrogenated by a non-naturally occurring chemical reaction. The hops beta acid can be essentially free of or substantially free of hydrogenated hops beta acid and/or hops acid. A non-naturally occurring chemical reaction is a chemical reaction that was conducted with the aid of chemical compound not found within Humulus lupulus, such as a chemical hydrogenation reaction conducted with high heat not normally experienced by Humulus lupulus in the wild and/or a metal catalyst.

A natural hops source can comprise from about 2% to about 12%, by weight of the hops source, of hops alpha acids. Hops extracts used in other contexts, such as in the brewing of beer, can comprise from about 15% to about 35%, by weight of the extract, of hops alpha acids. The hops extract desired herein can comprise less than about 10%, less than about 5%, less than about 1%, or less than about 0.5%, by weight of the extract, of hops alpha acids.

Hops oils can include terpene hydrocarbons, such as myrcene, humulene, caryophyllene, and/or mixtures thereof. The hops extract desired herein can comprise less than 5%, less than 2.5%, or less than 2%, by weight of the extract, of one or more hops oils.

Flavonoids present in the hops extract can include xanthohumol, 8-prenylnaringenin, isoxanthohumol, and/or mixtures thereof. The hops extract can be substantially free of, essentially free of, free of, or have less than 250 ppm, less than 150 ppm, and/or less than 100 ppm of one or more flavonoids.

As described in U.S. Pat. No. 5,370,863, hops acids have been previously added to oral care compositions. However, the oral care compositions taught by U.S. Pat. No. 5,370,863 only included up to 0.01%, by weight of the oral care composition. While not wishing to be bound by theory, it is believed that U.S. Pat. No. 5,370,863 could only incorporate a low amount of hops acids because of the bitterness of hops alpha acids. A hops extract with a low level of hops alpha acids would not have this concern.

The hops compound can be combined with or free from an extract from another plant, such as a species from genus Magnolia. The hops compounds can be combined with or free from triclosan.

The oral care composition can comprise from about 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops, such as hops beta acid, as described herein. The hops, such as the hops beta acid, can be provided by a suitable hops extract, the hops plant itself, or a synthetically derived compound. The hops, such as hops beta acid, can be provided as neutral, acidic compounds, and/or as salts with a suitable counter ion, such as sodium, potassium, ammonia, or any other suitable counter ion.

The hops can be provided by a hops extract, such as an extract from Humulus lupulus with at least 35%, by weight of the extract, of hops beta acid and less than 1%, by weight of the hops extract, of hops alpha acid. The oral care composition can comprise 0.01% to about 10%, greater than 0.01% to about 10%, from about 0.05%, to about 10%, from about 0.1% to about 10%, from about 0.2% to about 10%, from about 0.2% to about 10%, from about 0.2% to about 5%, from about 0.25% to about 2%, from about 0.05% to about 2%, or from greater than 0.25% to about 2%, of hops extract, as described herein.

### Flavor

The oral care composition comprise flavor. Many oral care compositions are formulated using one or more of mint flavors, such as peppermint, spearmint, wintergreen, and/or combinations thereof. Each mint flavor family comprises unique combinations of organic compounds that signal different flavor profiles.

For example, peppermint oil is comprised mostly of menthol, menthone, and menthyl acetate, and has lower amounts of the characterizing components of spearmint and/or wintergreen. Spearmint oil is mostly comprised of carvone and limonene, and contains low level of the characterizing components of peppermint and/or wintergreen. Wintergreen comprises primarily methyl salicylate, and lacks the unique components of peppermint and/or spearmint.

Surprisingly, as described herein, it has been discovered that the combinations of hops and wintergreen at certain amounts can lead to unpalatable flavor profile that can discourage use of oral care compositions comprising hops and aromatic esters, such as methyl salicylate found in wintergreen.

While not wishing to be bound by theory, it is believed that the combination of hops and methyl salicylate results in a uniquely unpalatable experience due at least in part to the bitterness of the hops being amplified instead of masked. Additionally, this combination of hops and aromatic esters leads to a sour and unpleasant experience for some users. Thus, only a minimal amount of aromatic esters can be used in combination with hops in oral care compositions

It was further determined that peppermint- and spearmint-based flavor systems can be combined with hops in oral care compositions to create palatable tastes.

Unpalatable flavors have been observed in combination with noticeable levels of methyl salicylate, a primary component of wintergreen and chemically distinct from peppermint and spearmint. As such, the present invention is directed towards oral care compositions comprising hops with less than a noticeable amount of methyl salicylate.

Thus, the flavor can include up to about 0.20%, up to about 0.15%, up to about 0.10%, by weight of the composition, of aromatic ester. The flavor can be essentially free of, substantially free of, or free of aromatic ester. Thus, the flavor can include up to about 0.20%, up to about 0.15%, up to about 0.10%, by weight of the composition, of aromatic ester. The flavor can be essentially free of, substantially free of, or free of aromatic ester.

The aromatic ester can comprise salicylate ester, alkyl salicylate, and/or combinations thereof. The alkyl salicylate can comprise methyl salicylate, ethyl salicylate, propyl salicylate, 2-methyl-propyl salicylate, butyl salicylate, pentyl salicylate, hexyl salicylate, heptyl salicylate, octyl salicylate, nonyl salicylate, and/or combinations thereof.

The flavor can include both traditional flavor compounds as well as sensates. Examples of some traditional flavor compounds that may be used in the flavor oral care compositions are mint oils, and components thereof, clove bud oil, cassia, sage, parsley oil, marjoram, lemon, orange, propenyl guaethol, heliotropine, cis-4-heptenal, diacetyl, methyl-ρ-tert-butyl phenyl acetate, 1-menthyl acetate, oxanone, α-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, α-terpineol, linalool, limonene, citral, neral, geranial, geraniol nerol, maltol, ethyl maltol, anethole, dihydroanethole, carvone, menthone, β-damascenone, ionone, γ-decalactone, γ-nonalactone, γ-undecalactone, isopulegol, piperitone, or combinations thereof. Generally suitable flavoring ingredients are chemicals with structural features and functional groups that are less prone to redox reactions. These include derivatives of flavor chemicals that are saturated or contain stable aromatic rings or ester groups.

The flavor can also comprise sensate. Sensate molecules such as cooling, warming, and tingling agents are useful to deliver signals to the user. Sensates can be present in an amount of from about 0.001% to about 2%, by weight of the oral care composition, alternatively from about 0.01% to about 1.75%, alternatively 0.1% to about 1.5%, and alternatively 0.5% to about 1.25%. Cooling sensate compounds can comprise menthol, particularly L-menthol, which is found naturally in peppermint and spearmint oils notably of Mentha piperita, Mentha arvensis L and Mentha viridis L. Other isomers of menthol (neomenthol, isomenthol and neoisomenthol) have somewhat similar, but not identical odor and taste, and may have, for instance, disagreeable odor and taste notes described as earthy, camphor, musty, etc. The biggest difference among the isomers is in their cooling potency. L-menthol provides the most potent cooling, by having the lowest cooling threshold of about 800 ppb, which is the concentration level where the cooling effect can be clearly recognized. At this level, there can be no cooling effect for the other isomers. For example, d-neomenthol is reported to have a cooling threshold of about 25,000 ppb and 1-neomenthol about 3,000 ppb.

Of the menthol isomers the 1-isomer occurs most widely in nature and is typically what is referred by the name menthol having coolant properties. L-menthol has the characteristic peppermint odor, has a clean fresh taste and exerts a cooling sensation when applied to the skin and mucosal surfaces.

Among synthetic coolants, many are derivatives of or are structurally related to menthol, for example containing the cyclohexane moiety, and derivatized with functional groups including carboxamide, ketal, ester, ether and alcohol. Examples include the ρ-menthanecarboxamide compounds such as N-ethyl-ρ-menthan-3-carboxamide, known commercially as “WS-3”, and others in the series such as WS-5 (N-ethoxycarbonylmethyl-ρ-menthan-3-carboxamide), WS-12 (1R*,2S*)-N-(4-Methoxyphenyl)-5-methyl-2-(1-methylethyl)cyclohexanecarboxamide] and WS-14 (N-tert-butyl-ρ-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”. Additional examples of synthetic coolants include alcohol derivatives such as 3-(1-menthoxy)-propane-1,2-diol known as TK-10, isopulegol (under the tradename Coolact P) and ρ-menthane-3,8-diol (under the tradename Coolact 38D) all available from Takasago Corp., Tokyo, Japan; menthone glycerol acetal known as MGA; menthyl esters such as menthyl acetate, menthyl acetoacetate, menthyl lactate known as Frescolat® supplied by Symrise A G, Holzminden, Germany, and monomenthyl succinate under the tradename Physcool from V. Mane FILS, Notre Dame, France. TK-10 is described in U.S. Pat. No. 4,459,425 to Amano et al. Other alcohol and ether derivatives of menthol are described in GB 1,315,626 and in U.S. Pat. Nos. 4,029,759; 5,608,119; and 6,956,139. WS-3 and other carboxamide cooling agents are described in U.S. Pat. Nos. 4,136,163; 4,150,052; 4,153,679; 4,157,384; 4,178,459 and 4,230,688.

Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide. Other N-substituted ρ-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N-((5-methyl-2-(1-methylethyl)cyclohexyl)carbonyl)alanine ethyl ester. Menthyl esters including those of amino acids such as glycine and alanine are disclosed e.g., in EP 310,299 and in U.S. Pat. Nos. 3,917,613; 3,991,178; 5,703,123; 5,725,865; 5,843,466; 6,365,215; and 6,884,903. Ketal derivatives are described, e.g., in U.S. Pat. Nos. 5,266,592; 5,977,166; and 5,451,404. Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H. R. Watson, et al. J. Soc. Cosmet. Chem. (1978), 29, 185-200 and R. Eccles, J. Pharm. Pharmacol., (1994), 46, 618-630 and phosphine oxides as reported in U.S. Pat. No. 4,070,496.

Some examples of warming sensates include ethanol; capsicum; nicotinate esters, such as benzyl nicotinate; polyhydric alcohols; capsicum powder; a capsicum tincture; capsicum extract; capsaicin; homocapsaicin; homodihydrocapsaicin; nonanoyl vanillyl amide; nonanoic acid vanillyl ether; vanillyl alcohol alkyl ether derivatives such as vanillyl ethyl ether, vanillyl butyl ether, vanillyl pentyl ether, and vanillyl hexyl ether; isovanillyl alcohol alkyl ethers; ethyl vanillyl alcohol alkyl ethers; veratryl alcohol derivatives; substituted benzyl alcohol derivatives; substituted benzyl alcohol alkyl ethers; vanillin propylene glycol acetal; ethyl vanillin propylene glycol acetal; ginger extract; ginger oil; gingerol; zingerone; or combinations thereof. Warming sensates are generally included in an oral care composition at a level of about 0.05% to about 2%, by weight of the oral care composition. The oral care composition can comprise from about 0.01% to about 5%, from about 0.4% to about 5%, from about 0.8% to about 4%, from about 1% to about 3.5%, or from about 1.5% to about 3%, by weight of the oral care composition, of the flavor. As described above, the flavor can be essentially free of, substantially free of, free of, or contain low levels of aromatic ester, such as alkyl salicylate and/or methyl salicylate.

### Fluoride Ion Source

The oral care composition can comprise fluoride, such as from a fluoride ion source. The fluoride ion source can comprise one or more fluoride containing compounds, such as stannous fluoride, sodium fluoride, titanium fluoride, calcium fluoride, calcium phosphate silicate fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, substantially free of, or free of a fluoride ion source.

### Tin Ion Source

The oral care composition of the present invention can comprise tin, such as from a tin ion source. The tin ion source can be any suitable compound that can provide tin ions in an oral care composition and/or deliver tin ions to the oral cavity when the dentifrice composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.5% to about 1.5%, or from about 0.3% to about 0.6%, by weight of the oral care composition, of a tin ion source.

### Ca Ion Source

The oral care composition of the present invention can comprise calcium, such as from a calcium ion source. The calcium ion source can be any suitable compound or molecule that can provide calcium ions in an oral care composition and/or deliver calcium ions to the oral cavity when the oral care composition is applied to the oral cavity. The calcium ion source can comprise a calcium salt, a calcium abrasive, and/or combinations thereof. In some cases, a calcium salt may also be considered a calcium abrasive or a calcium abrasive may also be considered a calcium salt.

The calcium ion source can comprise a calcium abrasive. The calcium abrasive can be any suitable abrasive compound that can provide calcium ions in an oral care composition and/or deliver calcium ions to the oral cavity when the oral care composition is applied to the oral cavity. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The calcium ion source can comprise a calcium salt, or a compound that can provide calcium ions in an oral care composition and/or deliver calcium ions to the oral cavity when the oral care composition is applied to the oral cavity that can not act as an abrasive. The calcium salt can comprise one or more calcium compounds, such as calcium chloride, calcium nitrate, calcium phosphate, calcium lactate, calcium oxalate, calcium oxide, calcium gluconate, calcium citrate, calcium bromide, calcium iodate, calcium iodide, hydroxyapatite, fluorapatite, calcium sulfate, calcium glycerophosphate, and/or combinations thereof.

The oral care composition can comprise from about 5% to about 70%, from about 10% to about 50%, from about 10% to about 60%, from about 20% to about 50%, from about 25% to about 40%, or from about 1% to about 50% of a calcium ion source.

### Buffering Agent

The oral care composition can comprise a buffering agent. The buffering agent can be a weak acid or base that can maintain a particular pH at a selected site in the oral cavity. For example, the buffering agent can maintain a pH at a tooth's surface to mitigate the impact of plaque acids produced by bacteria. The buffering agent can comprise a conjugate acid of an ion also present in the oral care composition. For example, if the calcium ion source comprises calcium carbonate, the buffering agent can comprise a bicarbonate anion (—HCO₃⁻). The buffering agent can comprise a conjugate acid/base pair, such as citric acid and sodium citrate.

Suitable buffering systems can include phosphate, citrate salts, carbonate/bicarbonate salts, a tris buffer, imidazole, urea, borate, and/or combinations thereof. Suitable buffering agents include bicarbonate salts, such as sodium bicarbonate, glycine, orthophosphate, arginine, urea, and or/combinations thereof.

The oral care composition can comprise from about 1% to about 30%, from about 5% to about 25% or from about 10% to about 20%, of one or more buffering agents.

### Biofilm Modifier

The oral care composition can comprise one or more biofilm modifiers. A biofilm modifier can comprise a polyol, an ammonia generating compound, and/or a glucosyltransferase inhibitor.

A polyol is an organic compound with more than one hydroxyl functional groups. The polyol can be any suitable compound that can weakly associate, interact, or bond to tin ions while the oral care composition is stored prior to use. The polyol can be a sugar alcohol, which area class of polyols that can be obtained through the hydrogenation of sugar compounds with the formula (CHOH)ₙH₂. The polyol can be glycerin, erythritol, xylitol, sorbitol, mannitol, butylene glycol, lactitol, and/or combinations thereof. The oral care composition can comprise 0.01% to about 70%, from about 5% to about 70%, from about 5% to about 50%, from about 10% to about 60%, from about 10% to about 25%, or from about 20% to about 80%, by weight of the oral care composition, of a polyol.

The ammonia generating compound can be any suitable compound that can generate ammonia upon delivery to the oral cavity. Suitable ammonia generating compounds include arginine, urea, and/or combinations thereof. The oral care composition can comprise from about 0.01% to about 10%, from about 1% to about 5%, or from about 1% to about 25% of one or more ammonia generating compounds.

The glucosyltransferase inhibitor can be any suitable compound that can inhibit a glucosyltransferase. Glucosyltransferases are enzymes that can establish natural glycosidic linkages. In particular, these enzymes break down poly- or oligosaccharide moieties into simple sugars for bacteria associated with dental caries. As such, any compound that can inhibit this process can help prevent dental caries. Suitable glucosyltransferase inhibitors include oleic acid, epicatechin, tannins, tannic acid, moenomycin, caspofungin, ethambutol, lufenuron, and/or combinations thereof. The oral care composition can comprise from about 0.001% to about 5%, from about 0.01% to about 2%, or about 1% of one or more glucosyltransferase inhibitors.

### Metal Ion Source

The oral care composition can comprise metal, such as from a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The trace metal source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of a metal ion source.

### Antibacterial Agents

The oral care composition can comprise one or more antibacterial agents. Suitable antibacterial agents include any molecule that provides antibacterial activity in the oral cavity. Suitable antibacterial agents include hops acids, tin ion sources, benzyl alcohol, sodium benzoate, menthylglycyl acetate, menthyl lactate, L-menthol, o-neomenthol, chlorophyllin copper complex, phenol, oxyquinoline, and/or combinations thereof.

The oral care composition can comprise from about 0.01% to about 10%, from about 1% to about 5%, or from about 0.5% to about 15% of an antibacterial agent.

### Bioactive Materials

The oral care composition can also include bioactive materials suitable for the remineralization of a tooth. Suitable bioactive materials include bioactive glasses, Novamin™, Recaldent™ hydroxyapatite, one or more amino acids, such as, for example, arginine, citrulline, glycine, lysine, or histidine, or combinations thereof. Suitable examples of compositions comprising arginine are found in U.S. Pat. Nos. 4,154,813 and 5,762,911, which are herein incorporated by reference in their entirety. Other suitable bioactive materials include any calcium phosphate compound. Other suitable bioactive materials include compounds comprising a calcium source and a phosphate source.

Amino acids are organic compounds that contain an amine functional group, a carboxyl functional group, and a side chain specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, and/or combinations thereof.

Bioactive glasses are comprising calcium and/or phosphate which can be present in a proportion that is similar to hydroxyapatite. These glasses can bond to the tissue and are biocompatible. Bioactive glasses can include a phosphopeptide, a calcium source, phosphate source, a silica source, a sodium source, and/or combinations thereof.

The oral care composition can comprise from about 0.01% to about 20%, from about 0.1% to about 10%, or from about 1% to about 10% of a bioactive material by weight of the oral care composition.

### Abrasive

The oral care composition can comprise a calcium abrasive, as described herein, and/or a non-calcium abrasive, such as bentonite, silica gel (by itself, and of any structure), precipitated silica, amorphous precipitated silica (by itself, and of any structure as well), hydrated silica, perlite, titanium dioxide, calcium pyrophosphate, dicalcium phosphate dihydrate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the oral care compositions to tailor the polishing characteristics of the target dentifrice formulation. The oral care composition can comprise from about 5% to about 70%, from about 10% to about 50%, from about 10% to about 60%, from about 20% to about 50%, from about 25% to about 40%, or from about 1% to about 50%, by weight of the oral care composition, of the non-calcium abrasive.

Alternatively, the oral care composition can be substantially free of, essentially free of, or free of silica, alumina, or any other non-calcium abrasive. The oral care composition can comprise less than about 5%, less than about 1%, less than about 0.5%, less than about 0.1%, or 0% of a non-calcium abrasive, such as silica and/or alumina.

### Water

The oral care composition of the present invention can be anhydrous, a low water formulation, or a high water formulation. In total, the oral care composition can comprise from 0% to about 99%, from about 5% to about 75%, about 20% or greater, about 30% or greater, or about 50% or greater by weight of the composition, of water. Preferably, the water is USP water.

In a high water oral care composition and/or toothpaste formulation, the oral care composition comprises from about 45% to about 75%, by weight of the composition, of water. The high water oral care composition and/or toothpaste formulation can comprise from about 45% to about 65%, from about 45% to about 55%, or from about 46% to about 54%, by weight of the composition, of water.

The water may be added to the high water formulation and/or may come into the composition from the inclusion of other ingredients.

In a low water oral care composition and/or toothpaste formulation, the oral care composition comprises from about 5% to about 45%, by weight of the composition, of water. The low water oral care composition can comprise from about 5% to about 35%, from about 10% to about 25%, or from about 20% to about 25%, by weight of the composition, of water. The water may be added to the low water formulation and/or may come into the composition from the inclusion of other ingredients.

In an anhydrous oral care composition and/or toothpaste formulation, the oral care composition comprises less than about 10%, by weight of the composition, of water. The anhydrous composition comprises less than about 5%, less than about 1%, or 0%, by weight of the composition, of water. The water may be added to the anhydrous formulation and/or may come into the composition from the inclusion of other ingredients.

A mouth rinse formulation comprises from about 75% to about 99%, from about 75% to about 95%, or from about 80% to about 95% of water.

The composition can also comprise other orally acceptable carrier materials, such as alcohol, humectants, polymers, surfactants, and acceptance improving agents, such as flavoring, sweetening, coloring and/or cooling agents.

The pH of the disclosed composition can be from about 4 to about 10, from about 7 to about 10, greater than 7 to about 10, greater than 8 to about 10, greater than 7, greater than 7.5, greater than 8, greater than 9, or from about 8.5 to about 10.

### Zinc Ion Source

The oral care composition can comprise zinc, such as from a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc ion source may be present in the total oral care composition at an amount of from about 0.01% to about 10%, from about 0.2% to about 1%, from about 0.5% to about 1.5%, or from about 0.3% to about 0.6%, by weight of the dentifrice composition.

### Polyphosphates

The oral care composition can comprise polyphosphate, such as from a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of, essentially free of, or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source.

### Humectants

The oral care composition can comprise one or more humectants, have low levels of a humectant, be essentially free of, be substantially free of, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants.

Suitable anionic surfactants include, for example, the water soluble salts of alkyl sulfates having from 8 to 20 carbon atoms in the alkyl radical and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants include sarcosinates, such as sodium lauroyl sarcosinate, taurates, sodium lauryl sulfoacetate, sodium lauroyl isethionate, sodium laureth carboxylate, and sodium dodecyl benzene sulfonate. Combinations of anionic surfactants can also be employed.

Another suitable class of anionic surfactants are alkyl phosphates. The surface active organophosphate agents can have a strong affinity for enamel surface and have sufficient surface binding propensity to desorb pellicle proteins and remain affixed to enamel surfaces. Suitable examples of organophosphate compounds include mono-, di- or triesters represented by the general structure below wherein Z₁, Z₂, or Z₃ may be identical or different with at least one being an organic moiety. Z₁, Z₂, or Z₃ can be selected from linear or branched, alkyl or alkenyl group of from 1 to 22 carbon atoms, optionally substituted by one or more phosphate groups; alkoxylated alkyl or alkenyl, (poly)saccharide, polyol or polyether group.

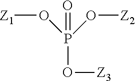

Some other agents include alkyl or alkenyl phosphate esters represented by the following structure:

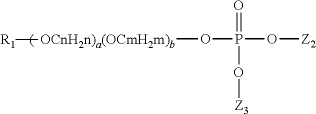

wherein R₁ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z and Z may be identical or different, each represents hydrogen, alkali metal, ammonium, protonated alkyl amine or protonated functional alkylamine, such as analkanolamine, or a R—(OCH2)(OCH)— group. Examples of suitable agents include alkyl and alkyl (poly)alkoxy phosphates such as lauryl phosphate; PPGS ceteareth-10 phosphate; laureth-1 phosphate; laureth-3 phosphate; laureth-9 phosphate; trilaureth-4 phosphate; C₁₂₋₁₈ PEG 9 phosphate: and sodium dilaureth-10 phosphate. The alkyl phosphate can be polymeric. Examples of polymeric alkyl phosphates include those containing repeating alkoxy groups as the polymeric portion, in particular 3 or more ethoxy, propoxy isopropoxy or butoxy groups.

Other suitable anionic surfactants are sarcosinates, isethionates and taurates, especially their alkali metal or ammonium salts. Examples include: lauroyl sarcosinate, myristoyl sarcosinate, palmitoyl sarcosinate, stearoyl sarcosinate oleoyl sarcosinate, or combinations thereof.

Other suitable anionic surfactants include sodium or potassium alkyl sulfates, such as sodium lauryl sulfate, acyl isethionates, acyl methyl isethionates, alkyl ether carboxylates, acyl alaninates, acyl gulatames, acyl glycinates, acyl sarconsinates, sodium methyl acyl taurates, sodium laureth sulfosuccinates, alpha olefin sulfonates, alkyl benze sulfonates, sodium lauroyl lactylate, sodium laurylglucosides hydroxypropyl sulfonate, and/or combinations.

Zwitterionic or amphoteric surfactants useful herein include derivatives of aliphatic quaternary ammonium, phosphonium, and Sulfonium compounds, in which the aliphatic radicals can be straight chain or branched, and one of the aliphatic substituents contains from 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577. Typical alkyl dimethyl betaines include decyl betaine or 2-(N-decyl-N,N-dimethylammonio) acetate, coco-betaine or 2-(N-coco-N,N-dimethyl ammonio)acetate, myristyl betaine, palmityl betaine, lauryl betaine, cetyl betaine, cetyl betaine, stearyl betaine, etc. The amidobetaines can be exemplified by cocoamidoethyl betaine, cocoamidopropyl betaine (CADB), and lauramidopropyl betaine. Other suitable amphoteric surfactants include betaines, sultaines, sodium laurylamphoacetates, alkylamphodiacetates, and/or combinations thereof.

Cationic surfactants useful in the present invention include, for example, derivatives of quaternary ammonium compounds having one long alkyl chain containing from 8 to 18 carbon atoms such as lauryl trimethylammonium chloride; cetyl pyridinium chloride; cetyl trimethyl-ammonium bromide; cetyl pyridinium fluoride or combinations thereof.

Nonionic surfactants that can be used in the compositions of the present invention include, for example, compounds produced by the condensation of alkylene oxide groups (hydrophilic in nature) with an organic hydrophobic compound which may be aliphatic or alkylaromatic in nature. Examples of suitable nonionic surfactants can include the Pluronics® which are poloxamers, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and combinations of such materials. Other suitable non-ionic surfactants includes alkyl glucamides, alkyl glucosides, and/or combinations thereof.

The one or more surfactants can also include one or more natural and/or naturally derived surfactants. Natural surfactants can include surfactants that are derived from natural products and/or surfactants that are minimally or not processed. Natural surfactants can include hydrogenated, non-hydrogenated, or partially hydrogenated vegetable oils, olus oil, Passiflora incarnata oil, candelilla cera, coco-caprylate, caprate, dicaprylyl ether, lauryl alcohol, myristyl myristate, dicaprylyl ether, caprylic acid, caprylic ester, octyl decanoate, octyl octanoate, undecane, tridecane, decyl oleate, oleic acid decylester, cetyl palmitate, stearic acid, palmitic acid, glyceryl stearate, hydrogenated, non-hydrogenated, or partially hydrogenated vegetable glycerides, Polyglyceryl-2 dipolyhydroxystearate, cetearyl alcohol, sucrose polystearate, glycerin, octadodecanol, hydrolyzed, partially hydrolyzed, or non-hydrolyzed vegetable protein, hydrolyzed, partially hydrolyzed, or non-hydrolyzed wheat protein hydrolysate, polyglyceryl-3 diisostearate, glyceryl oleate, myristyl alcohol, cetyl alcohol, sodium cetearyl sulfate, cetearyl alcohol, glyceryl laurate, capric triglyceride, coco-glycerides, lectithin, dicaprylyl ether, xanthan gum, sodium coco-sulfate, ammonium lauryl sulfate, sodium cocoyl sulfate, sodium cocoyl glutamate, polyalkylglucosides, such as decyl glucoside, cetearyl glucoside, cetyl stearyl polyglucoside, coco-glucoside, and lauryl glucoside, and/or combinations thereof. Natural surfactants can include any of the Natrue ingredients marketed by BASF, such as, for example, CegeSoft®, Cetiol®, Cutina®, Dehymuls®, Emulgade®, Emulgin®, Eutanol®, Gluadin®, Lameform®, LameSoft®, Lanette®, Monomuls®, Myritol®, Plantacare®, Plantaquat®, Platasil®, Rheocare®, Sulfopon®, Texapon®, and/or combinations thereof.

Other specific examples of surfactants include sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Additional surfactants desired include fatty acid salts of glutamate, alkyl glucoside, salts of taurates, betaines, caprylates, and/or mixtures thereof. The oral care composition can also be sulfate free.

The oral care composition can comprise one or more surfactants each at a level from about 0.01% to about 15%, from about 0.3% to about 10%, or from about 0.3% to about 2.5%, by weight of the oral care composition.

### Thickening Agents

The oral care composition can comprise one or more thickening agents. Thickening agents can be useful in the oral care compositions to provide a gelatinous structure that stabilizes the dentifrice and/or toothpaste against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, essentially free of, or substantially free of a copolymer of maleic anhydride and methyl vinyl ether.

The thickening agent can comprise one or more inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Prenylated Flavonoids

The oral care composition of the present invention can comprise prenylated flavonoid. Flavonoids are a group of natural substances found in a wide range of fruits, vegetables, grains, bark, roots, stems, flowers, tea, and wine. Flavonoids can have a variety of beneficial effects on health, such as antioxidative, anti-inflammatory, antimutagenic, anticarcinogenic, and antibacterial benefits. Prenylated flavonoids are flavonoids that include at least one prenyl functional group (3-methylbut-2-en-1-yl, as shown in Formula VIII), which has been previously identified to facilitate attachment to cell membranes. Thus, while not wishing to being bound by theory, it is believed that the addition of a prenyl group, i.e. prenylation, to a flavonoid can increase the activity of the original flavonoid by increasing the lipophilicity of the parent molecule and improving the penetration of the prenylated molecule into the bacterial cell membrane. Increasing the lipophilicity to increase penetration into the cell membrane can be a double-edged sword because the prenylated flavonoid will tend towards insolubility at high Log P values (high lipophilicity). Log P can be an important indicator of antibacterial efficacy.

As such, the term prenylated flavonoids can include flavonoids found naturally with one or more prenyl functional groups, flavonoids with a synthetically added prenyl functional group, and/or prenylated flavonoids with additional prenyl functional groups synthetically added.

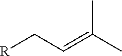

Formula VIII. Prenyl Function Group with R representing the other portions of the molecule

Other suitable functionalities of the parent molecule that improve the structure-activity relationship (e.g., structure-MIC relationship) of the prenylated molecule include additional heterocycles containing nitrogen or oxygen, alkylamino chains, or alkyl chains substituted onto one or more of the aromatic rings of the parent flavonoid.

Flavonoids can have a 15-carbon skeleton with at least two phenyl rings and at least one heterocyclic ring. Some suitable flavonoid backbones can be shown in Formula IX (flavone backbone), Formula X (isoflavan backbone), and/or Formula XI (neoflavonoid backbone).

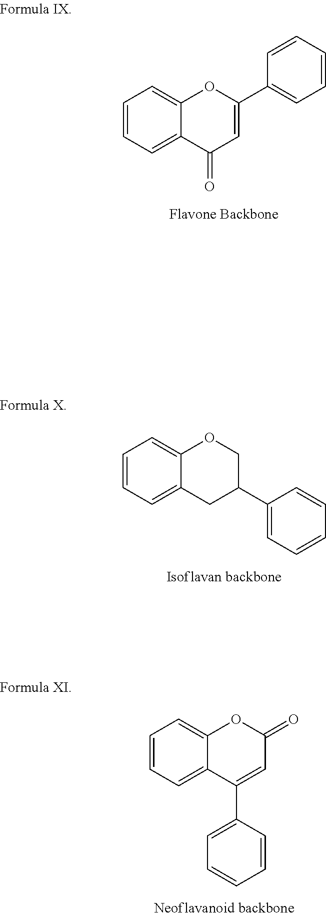 [SMILES: O=c1cc(-c2ccccc2)oc2ccccc12 ; c1ccc(C2COc3ccccc3C2)cc1 ; O=c1cc(-c2ccccc2)c2ccccc2o1]

Other suitable subgroups of flavonoids include anthocyanidins, anthoxanthins, flavanones, flavanonols, flavans, isoflavonoids, chalcones and/or combinations thereof.

Prenylated flavonoids can include naturally isolated prenylated flavonoids or naturally isolated flavonoids that are synthetically altered to add one or more prenyl functional groups through a variety of synthetic processes that would be known to a person of ordinary skill in the art of synthetic organic chemistry.

Other suitable prenylated flavonoids can include Bavachalcone, Bavachin, Bavachinin, Corylifol A, Epimedin A, Epimedin A1, Epimedin B, Epimedin C, Icariin, Icariside I, Icariside II, Icaritin, Isobavachalcone, Isoxanthohumol, Neobavaisoflavone, 6-Prenylnaringenin, 8-Prenylnaringenin, Sophoraflavanone G, (−)-Sophoranone, Xanthohumol, Quercetin, Macelignan, Kuraridin, Kurarinone, Kuwanon G, Kuwanon C, Panduratin A, 6-geranylnaringenin, Australone A, 6,8-Diprenyleriodictyol, dorsmanin C, dorsmanin F, 8-Prenylkaempferol, 7-O-Methylluteone, luteone, 6-prenylgenistein, isowighteone, lupiwighteone, and/or combinations thereof. Other suitable prenylated flavonoids include cannflavins, such as Cannflavin A, Cannflavin B, and/or Cannflavin C.

Preferably, the prenylated flavonoid has a high probability of having a MIC of less than about 25 ppm for S. aureus, a gram-positive bacterium. Suitable prenylated flavonoids include Bavachin, Bavachinin, Corylifol A, Icaritin, Isoxanthohumol, Neobavaisoflavone, 6-Prenylnaringenin, 8-Prenylnaringenin, Sophoraflavanone G, (−)-Sophoranone, Kurarinone, Kuwanon C, Panduratin A, and/or combinations thereof.

Preferably, the prenylated flavonoid has a high probability of having a MIC of less than about 25 ppm for E. coli, a gram-negative bacterium. Suitable prenylated flavonoids include Bavachinin, Isoxanthohumol, 8-Prenylnaringenin, Sophoraflavanone G, Kurarinone, Panduratin A, and/or combinations thereof.

Approximately 1000 prenylated flavonoids have been identified from plants. According to the number of prenylated flavonoids reported before, prenylated flavonones are the most common subclass and prenylated flavanols is the rarest sub-class. Even though natural prenylated flavonoids have been detected to have diversely structural characteristics, they have a narrow distribution in plants, which are different to the parent flavonoids as they are present almost in all plants. Most of prenylated flavonoids are found in the following families, including Cannabaceae, Guttiferae, Leguminosae, Moraceae, Rutaceae and Umbelliferae. Leguminosae and Moraceae, due to their consumption as fruits and vegetables, are the most frequently investigated families and many novel prenylated flavonoids have been explored. Humulus lupulus of the Cannabaceae include 8-prenylnaringenin and xanthohumol, which can play a role in the health benefits of beer.

The prenylated flavonoid can be incorporated through a hops extract, incorporated in a separately added extract, or added as a separate component of the oral care compositions disclosed herein.

Suitable prenylated flavonoids can have a particular octanol-water partitioning coefficient. The octanol-water partitioning coefficient can be used to predict the lipophilicity of a compound. Without wishing to being bound by theory, it is believed that compounds that fall within the ranges described herein will be able to enter and/or disrupt the primarily hydrophobic phospholipid bilayer that makes of the cell membrane of microorganisms. Thus, the octanol-water partitioning coefficient can be correlated to the antibacterial effect of prenylated flavonoids. Suitable prenylated flavonoids can have a log P of at least about 2, at least about 4, from about 2 to about 10, from about 4 to about 10, from about 4 to about 7, or from about 4 to about 7.

The oral care composition can comprise at least about 0.001%, from about 0.001% to about 5%, from about 0.01% to about 2%, from about 0.0001% to about 2%, or at least about 0.05% of prenylated flavonoid.

### Other Ingredients

The oral care composition can comprise a variety of other ingredients, such as sweeteners, colorants, preservatives, or other ingredients suitable for use in oral care compositions, as described below.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Combinations

A. An oral care composition comprising:
(a) hops; and
(b) flavor,
wherein the flavor comprises up to about 0.20%, by weight of the oral care composition, of aromatic ester.
B. The oral care composition as disclosed in A, wherein the aromatic ester comprises salicylate ester, preferably wherein the salicylate ester comprises alkyl salicylate, more preferred wherein the alkyl salicylate comprises methyl salicylate, ethyl salicylate, propyl salicylate, 2-methyl-propyl salicylate, butyl salicylate, pentyl salicylate, hexyl salicylate, heptyl salicylate, octyl salicylate, nonyl salicylate, or combinations thereof.
C. The oral care composition as disclosed in A or B, wherein the flavor comprises less than about 0.20%, by weight of the oral care composition, of aromatic ester.
D. The oral care composition as disclosed in any of A to C, wherein the hops comprises hops extract, Humulus lupulus extract, synthetically derived hops compounds, salts thereof, prodrugs thereof, or combinations thereof.
E. The oral care composition as disclosed in any of A to D, wherein the hops comprises hops alpha acid, hops iso-alpha acid, hops beta acid, hops oil, hops flavonoids, or combinations thereof.
F. The oral care composition as disclosed in E, wherein the hops beta acid comprises lupulone, adlupulone, colupulone, or combinations thereof.
G. The oral care composition as disclosed E or F, wherein the hops comprises at least about 35%, by weight of the hops, of hops beta acid.
H. The oral care composition as disclosed in any of E to G, wherein the hops comprises less than about 1%, by weight of the hops, of hops alpha acid.
I. The oral care composition as disclosed in any of A to H, wherein the wherein the oral care composition comprises from about 0.01% to about 10%, by weight of the oral care composition, of the hops.
J. The oral care composition as disclosed in any of A to I, wherein the oral care composition comprises tin, zinc, calcium or combinations thereof, preferably wherein the oral composition comprises tin, zinc, or combinations thereof.
K. The oral care composition as disclosed in J, wherein the tin comprises stannous fluoride, stannous chloride, or combinations thereof.
L. The oral care composition as disclosed in J or K, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.
M. The oral care composition as disclosed in any of J to L, wherein the calcium comprises calcium salt, calcium abrasive, or combinations thereof, preferably wherein the calcium abrasive comprises calcium carbonate, calcium pyrophosphate, or combinations thereof.
N. The oral care composition as disclosed in any of A to M, wherein the oral care composition comprises amino acid, preferably wherein the amino acid comprises arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, or combinations thereof.
O. The oral care composition as disclosed in any of A to N, wherein the oral care composition comprises silica abrasive.
P. The oral care composition as disclosed in any of A to 0, wherein the oral care composition comprises fluoride, preferably wherein the fluoride comprises stannous fluoride, sodium fluoride, potassium fluoride, amine fluoride, sodium monofluorophosphate, zinc fluoride, or combinations thereof.
Q. The oral care composition as disclosed in any of A to 0, wherein the oral care composition is free of fluoride.
R. The oral care composition as disclosed in any of A to Q for use in treatment of caries, bacteria, bad breath or combinations thereof.
S Method of reducing the bitter taste of compositions comprising hops comprising limiting the amount of aromatic ester to about 0.2% by weight of the composition.
T. The method of as disclosed in S, wherein the aromatic ester comprises salicylate ester, preferably wherein the salicylate ester comprises alkyl salicylate, more preferred wherein the alkyl salicylate comprises methyl salicylate, ethyl salicylate, propyl salicylate, 2-methyl-propyl salicylate, butyl salicylate, pentyl salicylate, hexyl salicylate, heptyl salicylate, octyl salicylate, nonyl salicylate, or combinations thereof.

### Examples

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention of the scope of the appended claims.

### Experimental Methods

### Panelist Use Data

The oral care compositions of TABLE 1 were prepared by combining one or more humectants, water, sweetener(s), metal ion sources, sodium gluconate, and/or flavor(s) to create a liquid mixture. The liquid mixture was homogenized at 25° C. for 2 minutes. Next, sodium hydroxide (50% solution) was added to the liquid mixture and the liquid mixture was homogenized at 25° C. for 2 minutes. A separate powder mixture was prepared by combining a portion of the abrasive and any thickening agents, such as xanthan gum, carrageenan gum, Gantrez, and/or hydroxyethyl cellulose. The powder mixture was then combined with the liquid mixture. Next, the surfactant, such as sodium lauryl sulfate, was added to the mixture. The contents were homogenized at 25° C. for 2 minutes. The hops extract was then combined, where appropriate, with the mixture and homogenized at 25° C. for 2 minutes. Finally, the remaining ingredients were combined with the mixture and homogenized at 25° C. for 2 minutes.

The toothpastes, once prepared, were used in a randomized, single-blinded, crossover study by a group of 15 panelists that evaluated the use experience across a number of attributes. Those attributes included assessments during brushing of bitter taste, cooling, and enjoyable overall flavor. Those attributes additionally included assessments after brushing of bitter taste, clean mouth feel, cooling, and medicinal taste. Product attribute assessment was conducted on the same toothpaste base with and without hops for two different flavors, a wintergreen- and a peppermint-based flavor. Thus, four formulas were rated by the panelists against the listed attributes using a 10-point scale where 1 would be a low value for that attribute and 10 would be high value for that attribute. The testing was conducted over an approximate two-week period where panelists could not use more than two products per day (once in the morning and once in the afternoon with at least a 3-hour rest period between uses) until all the products were evaluated.

### Degree-of-Difference Sensory Data

The oral care compositions of TABLE 3A and TABLE 3B were prepared by combining one or more humectants, water, sweetener(s), fluoride source, and/or peppermint flavor(s) to create a liquid mixture. The liquid mixture was homogenized at 25° C. for 2 minutes. A separate powder mixture was prepared by combining any thickening agents, such as carbomer and/or sodium carboxymethyl cellulose. The powder mixture was then combined with the liquid mixture. Next, the buffers were added to the liquid mixture and the liquid mixture was homogenized at 25° C. for 2 minutes. Next, the abrasives were added, and the mixture was homogenized at 25° C. for 2 minutes. Next, the surfactant, such as sodium lauryl sulfate, was added to the mixture. The contents were homogenized at 25° C. for 2 minutes. Finally, any remaining ingredients were combined with the mixture and homogenized at 25° C. for 2 minutes. The hops extract, methyl salicylate, and QS sorbitol was then combined, where appropriate, with the mixture and homogenized at 25° C. for 2 minutes. Toothpastes were then aged by storing at 40° C. for 21 days to reduce the intensity of flavors to those that would exist in a product at about six months of ambient aging following manufacturing. This gave a more realistic representation of what the end user would expect when they used the toothpaste.

Test samples were prepared and were given to trained and qualified graders in a “blind” format and were compared to a known control on a Five Point Degree of Difference (DOD) Scale. These grades, which indicate the degree of deviation from the control, determined the disposition of the product. The flavor aroma of the samples was compared for samples containing a dose response of methyl salicylate with a constant amount of hops and were assign DOD grades according to the following rubric.

| | |
|---|---|
| 1 - No difference. | No difference in character or intensity. |
| 2 - Slight difference. | Reasonably sure difference exists, though |
| | difference may be too subtle to accurately |
| | describe. |
| 3 - Moderate difference. | Definite difference; can describe difference |
| | with reasonable surety. |
| 4 - Large difference. | Moderate or large intensity differences or |
| | any character differences. |
| 5 - Extreme difference. | Large intensity and/or character differences. |

In this study, the graders had pre-loaded, 10 mL syringes with 1 g of dentifrice. The grader had 9 samples of the 0% methyl salicylate reference dentifrice and 9 samples of test dentifrices containing methyl salicylate. The grader would randomly choose a test dentifrice and compare that with the reference dentifrice. This was achieved by expunging the test and reference sample onto separate pieces of weigh paper, gently folding in half to slightly spread the dentifrice sample across the paper, and lightly sniffing the dentifrice. The examiner assigned a DOD score using the above rubric. After discarding the test and control weigh papers, the analyst would let their nose rest and repeat the process on another randomly chosen test dentifrice containing methyl salicylate and another reference dentifrice sample. The process was repeated until a DOD had been assigned to all test dentifrices. The average DOD was obtained for a panel of three expert graders.

TABLE 1
Oral Care Compositions for Panelist Use

| INGREDIENTS | Formula 1 | Formula 2 | Formula 3 | Formula 4 |
|---|---|---|---|---|
| Flavor Character | Wintergreen | Wintergreen | Peppermint | Peppermint |
| | | with Hops | | with Hops |

Toothpaste Base Ingredients

| | | | | |
|---|---|---|---|---|
| Stannous Fluoride | 0.45 | 0.45 | 0.45 | 0.45 |
| Sorbitol (70%) | 37.78 | 37.78 | 37.78 | 37.78 |
| Gantrez S-95 (35% soln.) | 5.71 | 5.71 | 5.71 | 5.71 |
| Zinc Lactate | 0.25 | 0.25 | 0.25 | 0.25 |
| Sodium Gluconate | 1.06 | 1.06 | 1.06 | 1.06 |
| Hops Beta Acid Extract | | 1.11 | | 1.11 |
| Sodium Saccharin | 0.80 | 0.80 | 0.80 | 0.80 |
| Z109 Amorphous Silica | 15.0 | 15.0 | 15.0 | 15.0 |
| Hydroxyethylcellulose | 0.72 | 0.72 | 0.72 | 0.72 |
| Carrageenan | 1.08 | 1.08 | 1.08 | 1.08 |
| Xanthan Gum, NF | 0.54 | 0.54 | 0.54 | 0.54 |
| Xylitol | 3.00 | 3.00 | 3.00 | 3.00 |
| Sodium lauryl sulfate (27.9%) | 5.00 | 5.00 | 5.00 | 5.00 |
| Sodium Hydroxide (50%) | 1.40 | 1.40 | 1.40 | 1.40 |
| Dye | 0.20 | 0.20 | 0.20 | 0.20 |
| TiO₂ | 0.50 | 0.50 | 0.50 | 0.50 |
| USP Water | QS to 100 | QS to 100 | QS to 100 | QS to 100 |

Flavoring Ingredients

| | | | | |
|---|---|---|---|---|
| Peppermint Base Flavor | 0.48 | 0.48 | 1.2 | 1.2 |
| Methyl Salicylate | 0.72 | 0.72 | | |

The compositions in Table 1 were assessed by panelists for the during brushing and after brushing attributes. Formula 1 was a wintergreen flavor without hops. Formula 2 was a wintergreen flavor with hops. Formula 3 was a peppermint flavor without hops. Formula 4 was a peppermint flavor with hops. In other aspects, the formulas were identical. The peppermint base flavor was reduced in order to replace it with the wintergreen flavor chemical, methyl salicylate.

TABLE 2
Oral Care Evaluations for Panelist Use Compositions

| | Formula | Formula | Formula | Formula |
|---|---|---|---|---|
| | 1 | 2 | 3 | 4 |
| During Brushing Bitter Taste | 2.11 | 3.75* | 1.87 | 1.66 |
| During Brushing Cooling | 3.50 | 4.30 | 6.07* | 6.72* |
| During Brushing Enjoyable Flavor | 5.20 | 5.18 | 5.23 | 6.47* |
| After Brushing Bitter Taste | 1.92 | 3.96* | 2.09 | 1.88 |
| After Brushing Clean Mouth Feel | 3.73 | 4.51 | 6.38* | 5.59* |
| After Brushing Cooling | 3.08 | 3.82 | 6.59* | 7.30* |
| After Brushing Medicinal Taste | 3.26 | 5.13* | 2.25 | 1.95 |

*Significantly different within each attribute assessed, p < 0.05 in student's t-test.

Two key attributes were elevated in Formula 2 (hops with wintergreen), namely during and after brushing bitter taste, as well as after brushing medicinal taste (an attribute closely associated with bitter experience). Formula 2 (hops with wintergreen) produced negative comments in taste related attributes. Bitterness, metallic and medicinal taste increased in intensity from hops formula containing methyl salicylate (Formula 2) vs. non-methyl-salicylate flavor (Formula 4). Even at a high hops level, the non-methyl-salicylate flavor (Formula 4) demonstrated many favorable ratings. Interestingly, subjects commented on the sour flavor experience in Formula 2 (wintergreen/methyl salicylate), which developed soon after the products were made. In total, the methyl salicylate found within wintergreen flavors exacerbated the unpleasant aroma and taste of hops in oral care compositions.

The level of menthol and peppermint in the base flavor composition were reduced in the wintergreen flavor in order to add methyl salicylate while keeping the overall flavor percentage in the composition the same. Consistent with this change were evaluable differences in the cooling of each flavor because menthol is a primary driver of cooling during and immediately after brushing. In a sense, this internal control on the flavor experience helped to validate the responses of the panelists with respect to their ability to distinguish between flavor character changes.

TABLE 3A
Oral Care Compositions in Degree-of-Difference Testing

| | Formula | Formula | Formula | Formula | Formula |
|---|---|---|---|---|---|
| | A, Ref | B | C | D | E |
| Sorbitol 70% Solution | 62.12 | 62.11 | 62.07 | 62.02 | 61.97 |
| Carboxymethyl Cellulose | 0.72 | 0.72 | 0.72 | 0.72 | 0.72 |
| Tribasic Sodium | 1.06 | 1.06 | 1.06 | 1.06 | 1.06 |
| Phosphate | | | | | |
| Sodium Phosphate | 0.40 | 0.40 | 0.40 | 0.40 | 0.40 |
| Monobasic | | | | | |
| Carbomer | 0.29 | 0.29 | 0.29 | 0.29 | 0.29 |
| Titanium Dioxide | 0.50 | 0.50 | 0.50 | 0.50 | 0.50 |
| Water | 10.72 | 10.72 | 10.72 | 10.72 | 10.72 |
| Silica | 14.40 | 14.40 | 14.40 | 14.40 | 14.40 |
| Sodium Lauryl Sulfate | 3.84 | 3.84 | 3.84 | 3.84 | 3.84 |
| 28% Solution | | | | | |
| Sodium Fluoride | 0.243 | 0.243 | 0.243 | 0.243 | 0.243 |
| Saccharin | 0.380 | 0.380 | 0.380 | 0.380 | 0.380 |
| Hops Beta Acid Extract | 0.500 | 0.500 | 0.500 | 0.500 | 0.500 |
| Spearmint Flavor | 1.000 | 1.000 | 1.000 | 1.000 | 1.000 |
| Methyl Salicylate | 0 | 0.010 | 0.050 | 0.100 | 0.150 |

TABLE 3B
Oral Care Compositions in Degree-of-Difference Testing

| | Formula | Formula | Formula | Formula | Formula |
|---|---|---|---|---|---|
| | F | G | H | I | J |
| Sorbitol 70% Solution | 62.12 | 62.11 | 62.07 | 62.02 | 61.97 |
| Carboxymethyl Cellulose | 0.72 | 0.72 | 0.72 | 0.72 | 0.72 |
| Tribasic Sodium | 1.06 | 1.06 | 1.06 | 1.06 | 1.06 |
| Phosphate | | | | | |
| Sodium Phosphate | 0.40 | 0.40 | 0.40 | 0.40 | 0.40 |
| Monobasic | | | | | |
| Carbomer | 0.29 | 0.29 | 0.29 | 0.29 | 0.29 |
| Titanium Dioxide | 0.50 | 0.50 | 0.50 | 0.50 | 0.50 |
| Water | 10.72 | 10.72 | 10.72 | 10.72 | 10.72 |
| Silica | 14.40 | 14.40 | 14.40 | 14.40 | 14.40 |
| Sodium Lauryl Sulfate | 3.84 | 3.84 | 3.84 | 3.84 | 3.84 |
| 28% Solution | | | | | |
| Sodium Fluoride | 0.243 | 0.243 | 0.243 | 0.243 | 0.243 |
| Saccharin | 0.380 | 0.380 | 0.380 | 0.380 | 0.380 |
| Hops Beta Acid Extract | 0.500 | 0.500 | 0.500 | 0.500 | 0.500 |
| Spearmint Flavor | 1.000 | 1.000 | 1.000 | 1.000 | 1.000 |
| Methyl Salicylate | 0.200 | 0.250 | 0.300 | 0.400 | 0.500 |

The challenge, therefore, is to define the level of methyl salicylate that negatively impacts the flavor experience. By using an expert panel to detect when methyl salicylate becomes a definable experience, we believe we can define a maximum threshold under which the flavor experience is minimally impacted and above which it is unpleasantly impacted. The formulas in Tables 3A and 3B were assessed using the DOD method by a team of expert graders. The test compositions, Formulas B-J, containing methyl salicylate were assessed against a reference composition, Formula A.

TABLE 4
Degree-of-Difference Evaluations from Expert Sensory Panelist

| | | | | |
|---|---|---|---|---|
| % Methyl | Flavor | Flavor | Flavor | Expert |
| Sal | Expert 1 | Expert 2 | Expert 3 | Average |

| | |
|---|---|
| 0 | Reference Condition |

| | | | | |
|---|---|---|---|---|
| 0.01 | 1.5 | 2.0 | 2.0 | 1.83 |
| 0.05 | 1.5 | 2.0 | 2.0 | 1.83 |
| 0.10 | 2.5 | 2.0 | 2.0 | 2.17 |
| 0.15 | 2.0 | 2.0 | 3.0 | 2.33 |
| 0.20 | 3.0 | 2.0 | 2.0 | 2.33 |

Transition from slight, indescribable to
definable difference at ca. 0.2%

| | | | | |
|---|---|---|---|---|
| 0.25 | 3.0 | 3.0 | 4.0 | 3.33 |
| 0.30 | 2.75 | 2.0 | 4.0 | 2.32 |
| 0.40 | 2.0 | 3.0 | 4.0 | 3.00 |
| 0.50 | 3.0 | 3.0 | 4.0 | 3.33 |

Methyl salicylate is a potent flavor experience and was noticeable by degree-of-difference measurements among expert flavorists at relatively low levels in comparison to the amount used in the consumer panel from Table 1. However, it was not identifiable as a distinct and definable sensory experience in combination with hops until higher levels, ca. 0.2%, as illustrated in Table 4. The amount of methyl salicylate required for a definable wintergreen experience was significantly less than that for the dentifrices used with the brushing panel in Table 1 and gave a conservative assessment of when the bitter/sour experience will be experienced in oral care compositions comprising both methyl salicylate and hops. The results in Table 4 illustrate that low levels of methyl salicylate can be included without negatively impacting the flavor experience; however, the flavor experience will be made unpleasant with levels of methyl salicylate above 0.2% in combination with hops.

TABLE 5
Hops Beta Acids Extract Specification

| Ingredient | Amount (wt %) |
|---|---|
| Hops Beta Acids | 45 ± 2 |
| Hops Alpha Acids | 0.4 ± 0.3 |
| Hops oils | 1.5 ± 0.5 |
| Propylene Glycol | 20 ± 15 |
| Water | <8% |
| pH | 11 ± 0.5 |

TABLE 5 describes the hops beta acid extract provided by Hopsteiner®. Since the hops beta acids are provided as an extract, there can be some variability in the amounts of certain ingredients. However, the extract comprises approximately 45%, by weight of the extract, of the hops beta acids and approximately 0.4%, by weight of the extract, of hops alpha acids. This is dramatically different to previous hops extracts which typically have more hops alpha acids than hops beta acids. Other minor ingredients may be present in the Hops Beta Acid extract.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
(a) hops; and
(b) flavor,
wherein the flavor comprises up to about 0.20%, by weight of the oral care composition, of aromatic ester.

2. The oral care composition of claim 1, wherein the aromatic ester comprises salicylate ester.

3. The oral care composition of claim 2, wherein the salicylate ester comprises alkyl salicylate.

4. The oral care composition of claim 3, wherein the alkyl salicylate comprises methyl salicylate, ethyl salicylate, propyl salicylate, 2-methyl-propyl salicylate, butyl salicylate, pentyl salicylate, hexyl salicylate, heptyl salicylate, octyl salicylate, nonyl salicylate, or combinations thereof.

5. The oral care composition of claim 1, wherein the flavor comprises less than about 0.20%, by weight of the oral care composition, of aromatic ester.

6. The oral care composition of claim 1, wherein the hops comprises hops extract, Humulus lupulus extract, synthetically derived hops compounds, salts thereof, prodrugs thereof, or combinations thereof.

7. The oral care composition of claim 1, wherein the hops comprises hops alpha acid, hops iso-alpha acid, hops beta acid, hops oil, hops flavonoids, or combinations thereof.

8. The oral care composition of claim 7, wherein the hops beta acid comprises lupulone, adlupulone, colupulone, or combinations thereof.

9. The oral care composition of claim 7, wherein the hops comprises at least about 35%, by weight of the hops, of hops beta acid.

10. The oral care composition of claim 9, wherein the hops comprises less than about 1%, by weight of the hops, of hops alpha acid.

11. The oral care composition of claim 1, wherein the oral care composition comprises from about 0.01% to about 10%, by weight of the oral care composition, of the hops.

12. The oral care composition of claim 1, wherein the oral care composition comprises tin, zinc, or combinations thereof.

13. The oral care composition of claim 12, wherein the tin comprises stannous fluoride, stannous chloride, or combinations thereof.

14. The oral care composition of claim 12, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.

15. The oral care composition of claim 1, wherein the oral care composition comprises amino acid.

16. The oral care composition of claim 15, wherein the amino acid comprises arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutonic acid, diaminoproprionic acid, salts thereof, or combinations thereof.

17. The oral care composition of claim 1, wherein the oral care composition comprises calcium.

18. The oral care composition of claim 17, wherein the calcium comprises calcium salt, calcium abrasive, or combinations thereof.

19. The oral care composition of claim 18, wherein the calcium abrasive comprises calcium carbonate, calcium pyrophosphate, or combinations thereof.

20. The oral care composition of claim 1, wherein the oral care composition comprises silica abrasive.

21. The oral care composition of claim 1, wherein the oral care composition is free of fluoride.

22. The oral care composition of claim 1, wherein the oral care composition comprises fluoride.

23. The oral care composition of claim 22, wherein the fluoride comprises stannous fluoride, sodium fluoride, potassium fluoride, amine fluoride, sodium monofluorophosphate, zinc fluoride, or combinations thereof.
